(19) United States
(12) Patent Application Publication
(10) Pub. No.: US 2026/0295641 A1
(43) Pub. Date: Oct. 1, 2026

(54) BASE STATION FOR A FLOOR CLEANER

(71) Applicant: BSH Hausgeräte GmbH, München (DE)

(72) Inventors: Maximilian FREMEREY, Saal (DE); Hari Teja SADHANALA, Bad Neustadt (DE)

(21) Appl. No.: 19/633,043

(22) Filed: Mar. 30, 2026

(30) Foreign Application Priority Data
Mar. 31, 2025 (DE) 102025112505.8

(51) Int. Cl. B08B 1/40; A47L 11/40; A61L 2/18; A61L 2/24; A61L 103/50

(52) U.S. Cl. CPC B08B 1/40; A47L 11/4091; A61L 2/18; A61L 2/24; A47L 2201/028; A61L 2103/50; A61L 2202/14; A61L 2202/15; A61L 2202/16; A61L 2202/17

(57) ABSTRACT

A method for cleaning and/or disinfecting a mopping textile of a cleaning appliance in a base station. A defined amount of cleaning or disinfecting fluid is supplied, in a pulsating feed, to the mopping textile for a predetermined first period of time. The mopping textile is moved over mechanical structures in order to squeeze dirt and also excess fluid out of the mopping textile and to discharge the dirt and excess fluid into a collecting facility. The fluid is stopped and the movement of the mopping textile over the mechanical structures is continued for a predetermined second period of time. The first period of time is shorter than the subsequent second period of time. There is also disclosed a cleaning and/or disinfecting station for cleaning and/or disinfecting a mopping textile of a cleaning appliance, and a cleaning and/or disinfecting system with such a cleaning and/or disinfecting station.

### CROSS-REFERENCE TO RELATED APPLICATION

This application claims the priority, under 35 U.S.C. § 119, of German Patent Application DE 10 2025 112 505.8, filed Mar. 31, 2025; the prior application is herewith incorporated by reference in its entirety.

### FIELD AND BACKGROUND OF THE INVENTION

The present invention relates to a method for cleaning and/or disinfecting a mopping textile of a cleaning appliance in a cleaning and/or disinfecting station. The invention further relates to a cleaning and/or disinfecting station for a corresponding system.

Currently known cleaning robots can vacuum as well as wet-mop and usually use flat mopping textiles made of natural and/or synthetic fibers. These mopping textiles first absorb water and then dirt from the floor during cleaning. To improve the cleaning effect, many of these robots are equipped with an actuator that sets the mopping textile in rotation or oscillation. After a certain period of use, however, the mopping textile becomes saturated with dirt, which means that it no longer cleans, but simply spreads the dirt. Modern cleaning robots therefore have a base station with a cleaning and/or disinfecting function, which enables automatic cleaning and, if necessary, disinfection of the mopping textile. In the base station, the mopping textile is treated by mechanical structures in combination with a fluid supply.

A similar method of this type is disclosed in Chinese published patent application CN 1 17 694 779 A. There, a cleaning part is first supplied with fluid, then the supply of fluid is stopped, and the cleaning part continues to move, while a wiping mechanism removes the dirt. The contaminated fluid is transferred to a collecting container by a vacuum system and finally drawn away. This method reduces water consumption and increases cleaning efficiency, but does not take into account targeted control of fluid supply or mechanically squeezing out dirt.

### SUMMARY OF THE INVENTION

It is accordingly an object of the invention to provide a base station for a floor cleaner which overcomes a variety of disadvantages associated with the heretofore-known devices and methods of this general type and which provides for an improved method and also an apparatus that enable efficient cleaning and/or disinfection of a mopping textile and minimize fluid consumption. In particular, the amount of fluid should be reduced and at the same time the cleaning efficiency should be maximized.

With the above and other objects in view there is provided, in accordance with the invention, a method for cleaning and/or disinfecting a mopping textile of a cleaning appliance in a base station, the method comprising the following:
a) supplying, in a pulsating supply of fluid, a defined amount of a cleaning or disinfecting fluid to the mopping textile for a predetermined first period of time;
b) moving the mopping textile over mechanical structures to squeeze dirt and excess fluid out of the mopping textile and discharging the dirt and excess fluid into a collecting facility; and
c) subsequently stopping the supply of fluid while continuing a movement of the mopping textile over the mechanical structures for a predetermined second period of time;
   wherein the first period of time is shorter than the second period of time.

The fluid is supplied via a fluid supply facility, which can be designed as a membrane or peristaltic pump. Alternatively, a pressure control via a valve system can be used in order to dose the quantity of fluid more precisely. The fluid is drawn from a reservoir and conveyed to the mopping textile.

The collecting facility is, for example, a watertight trough or a container, the inner wall of which is designed to be inclined in relation to a horizontal in order to collect fluid in a targeted manner at a lowest point. For example, the inner wall, in particular the trough base, is inclined about at least one axis.

In step c), the mopping textile is squeezed out further after the supply of fluid has ended in order to achieve maximum removal of water. Due to the repeated contact with the mechanical structures, dirt is removed from the textile and excess fluid is guided into the collecting facility.

The expressions "supply in a pulsing manner," or “pulsating supply”, or “pulsating feed” mean the intermittent fluid application at fixed intervals in order to achieve a resource-saving moistening. The term "defined amount" means the exactly dosed amount of fluid, which can be adapted to the degree of contamination and the area to be cleaned.

The cleaning or disinfecting fluid is a fluid that is used to remove dirt or to reduce the accumulation or growth of germs on the mopping textile. It can consist of water, a mixture of water and cleaning agent (cleaning fluid) or a mixture of water and disinfectant (disinfecting fluid), for example sodium hypochlorite.

The mopping textile is the cleaning element of the cleaning appliance, which absorbs dirt and fluid during the cleaning process. It can be made of synthetic or natural fibers and is designed either as a flat mopping cloth or as a mop. The cleaning appliance can have a plurality of such mopping textiles that can be cleaned using the method described herein. The mopping textile can rotate or oscillate about a substantially vertical axis, i.e. the z axis.

A first period of time comprises the fluid supply and the cleaning process, while a second period of time describes the squeezing process. Experiments have shown that the second period of time should be at least five times longer than the first period of time in order to ensure efficient removal of water. Accordingly, the second period of time is preferably at least five times the first period of time.

A mechanical structure is a fixed or movable component within a cleaning region of the cleaning station that comes into contact with the mopping textile and exerts mechanical effects on it. The mechanical structure can be designed as a fixed or movable projection having a triangular cross-section. The mechanical structure can be designed, for example, as a projection having a triangular cross-section in order to remove dirt and excess fluid from the mopping textile.

The cleaning region is the section of the base station, i.e., the cleaning and/or disinfecting station, in which the mopping textile is treated with cleaning or disinfecting fluid and also squeezed out mechanically. The cleaning region is configured so as to receive the mopping textile. The mechanical structures are provided in the cleaning region.

The expression "moving the mopping textile" is to be understood as the process in which the mopping textile is guided over the mechanical structures during cleaning. This can be done by moving the entire cleaning appliance or by an independent rotation or oscillation of the mopping textile. In this sense, the mopping textile is moved in a rotating or oscillating manner during step b) and/or step c). When the mopping textile is moved, fluid is squeezed out of the mopping textile by the mechanical structures. The mopping textile is therefore subjected to mechanical stress, for example due to pressure or friction, so that fluid is removed from the mopping textile. The mopping textile may be moved by an actuator of the cleaning appliance.

The rotating movement describes the continuous rotational movement of the mopping textile about a central axis. This movement serves to increase the cleaning efficiency by guiding the textile evenly over the surface to be cleaned. The oscillating movement is a reciprocating movement of the mopping textile along an axis. It supports the removal of stuck dirt by repeatedly moving the textile over the floor area or mechanical structures. These types of movement can also be used advantageously for cleaning or disinfecting the mopping textile in the afore-mentioned method.

During this process, the excess fluid and also the dirt particles pass into the collecting facility. The collecting facility receives the squeezed-out fluid and also the dirt particles. The collecting facility is preferably watertight and can be designed to be inclined in order to guide the collected fluid in a controlled manner into an outlet or a predetermined collection point. The outlet is preferably arranged at the lowest point of the collecting facility in order to efficiently remove fluid by gravity.

During step a), step b) and/or step c), the fluid that is collected in the collecting facility is preferably discharged via a fluid draining facility, for example a pump or a vacuum system, into a wastewater collecting container. The wastewater collecting container is an, in particular closed, tank or container in which the dirty fluid that is removed from the mopping textile is collected. It prevents uncontrolled leakage of fluid.

According to a particular embodiment, a degree of contamination of the discharged fluid is detected. The degree of contamination describes the amount and concentration of dirt particles or other impurities in the collected fluid. The degree of contamination of the fluid that is discharged into the wastewater collecting container can be detected by an optical turbidity measurement or a conductivity measurement.

Steps a) to c) can be repeated until the detected degree of contamination is below a limit value or threshold value. The number or intensity of the pulse cycles can be adjusted accordingly. In this sense, the pulse frequency of the fluid supply can be dynamically adapted to the degree of contamination. The limit value of the degree of contamination is a defined threshold value that determines at which point the cleaning cycles are continued or ended. Only when the measured contamination falls below this limit value is the cleaning process completed. In addition, the two periods of time can be adapted with each additional cleaning cycle. Further, the amount of fluid used can be adjusted and varied during each fluid supply.

With the above and other objects in view there is also provided, in accordance with the invention, a cleaning and/or disinfecting station, i.e., a base station, for a cleaning and/or disinfecting system. The base station comprises a fluid supply facility for supplying, in a pulsing feed, a defined amount of cleaning or disinfecting fluid to the mopping textile, a collecting facility for collecting fluid, and a cleaning region having mechanical structures which are designed to squeeze out dirt and also excess fluid by mechanical processing of the mopping textile and to discharge the dirt and excess fluid into the collecting facility. The mechanical processing is moved over the mechanical structures by moving the mopping textile, so that dirt and also excess fluid are squeezed out when contacting the mechanical structures and flow into the collecting facility.

The cleaning and/or disinfecting station is the base station of a cleaning or disinfecting system in which the mopping textile is cleaned and/or disinfected after a cleaning process. It can include additional functions such as the automatic refilling of fluids or the emptying of wastewater tanks.

The fluid supply facility is a component of the cleaning station that sucks the cleaning or disinfecting fluid from a reservoir and conveys it in a pulsing manner to the mopping textile. The fluid supply facility can be designed as a pump, for example a membrane or peristaltic pump, and is regulated in a pulsing manner by a control unit.

The collecting facility preferably has an outlet for discharging the collected fluid into the wastewater collecting container. The outlet is preferably fluidically connected to a wastewater collecting container, wherein a fluid draining facility is provided and is designed so as to discharge fluid that is collected in the collecting facility into the wastewater collecting container via the outlet. The fluid draining facility is a pump or a vacuum system that transports the collected dirty fluid from the collecting facility into a closed wastewater collecting container. Alternatively, a gravity process with supplementary pump control can be used in order to ensure a particularly energy-saving discharge.

Further preferably, the wastewater collecting container is designed as a closed system in order to prevent the escape of fluid vapors. The escape of fluid vapors refers to the uncontrolled evaporation of fluid components, in particular cleaning or disinfecting fluids.

According to one exemplary embodiment, the mechanical structures are designed as projections having a substantially triangular cross-sectional shape, wherein the projections come into contact with the mopping textile. The projections with the triangular cross-section squeeze the mopping textile out during movement, wherein the flanks of the projections discharge the squeezed-out fluid into the collecting facility. In particular, the projections are designed to be tapered. The mechanical structures can be mounted in a fixed or movable manner in order to enable an optimized adaptation to different mopping textiles. An embodiment of the mechanical structures that differs from a triangular cross-sectional shape is also conceivable. The shape and geometry of the mechanical structures can be adapted to the installation space conditions and the design of the mopping textile.

At least one of the mechanical structures, in particular at least one of the afore-mentioned projections, has one or more openings for supplying fluid, wherein water, cleaning fluid or disinfecting fluid can be supplied via the opening or openings. An opening for supplying fluid is to be understood as a passage within the mechanical structure, through which cleaning or disinfecting fluid is applied to the mopping textile. As a result, the fluid can be supplied in a targeted manner to specific contact points.

The number of mechanical structures is at least four and is an even number, so that they are evenly distributed under the mopping textile.

In one exemplary embodiment, the fluid supply facility is provided for providing and supplying water or cleaning fluid, wherein a disinfecting fluid supply facility is additionally provided for providing and supplying disinfecting fluid to the mopping textile. In other words, the cleaning and/or disinfecting station has two supply facilities, as a result of which the cleaning and/or disinfecting system can perform both cleaning functions and disinfecting functions. This means that the cleaning and/or disinfecting system can be used in a flexible manner.

The disinfecting fluid supply facility is an apparatus for providing and supplying a disinfecting fluid to the mopping textile. The disinfecting fluid supply facility can be designed as a unit for in-situ generation of a disinfecting fluid, wherein sodium hypochlorite is preferably used as the disinfecting fluid.

A proposed cleaning and/or disinfecting system comprises a cleaning and/or disinfecting station in accordance with the above embodiments and also a cleaning appliance which is equipped with a movable, in particular rotating or oscillating, mopping textile. The system is designed as a modular system and can be adapted to different cleaning appliances having rotating or oscillating mopping textiles.

A cleaning appliance is an apparatus for floor cleaning that uses a mopping textile to absorb dirt. The cleaning appliance can be designed, for example, as a cleaning robot, in particular as a mopping robot, preferably as a suction and mopping robot. Cleaning robots usually combine a vacuuming function with a wet cleaning and have at least one mopping textile, which is moistened with a cleaning fluid during operation in order to absorb dirt from the floor. In this case, the mopping textile can be moved either in a stationary manner or in rotation or oscillation in order to improve the cleaning performance.

The cleaning and/or disinfecting station is designed, for example, as a base station of the system, to which the cleaning robot automatically returns after a predefined cleaning process. The mopping textile is cleaned and/or disinfected at the base station, so that, following this process, the cleaning robot can work with a cleaned and/or disinfected mopping textile. In addition to the hygienization, i.e. the cleaning and/or disinfection, the base station can also perform other functions such as the automatic emptying of the dust container of the cleaning robot or the refilling of fresh water. By integrating the cleaning and/or disinfecting station into the base station, a fully automated cleaning system is created that enables continuous and efficient floor cleaning without manual intervention.

Other features which are considered as characteristic for the invention are set forth in the appended claims.

Although the invention is illustrated and described herein as embodied in a base station for a floor cleaner, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE FIGURES

FIG. 1 shows a highly schematic illustration of a cleaning and/or disinfecting system having a cleaning and/or disinfecting station; and

FIG. 2 shows a block diagram to illustrate a method for cleaning and/or disinfecting a mopping textile of a cleaning appliance in the cleaning and/or disinfecting station according to FIG. 1.

### DETAILED DESCRIPTION OF THE INVENTION

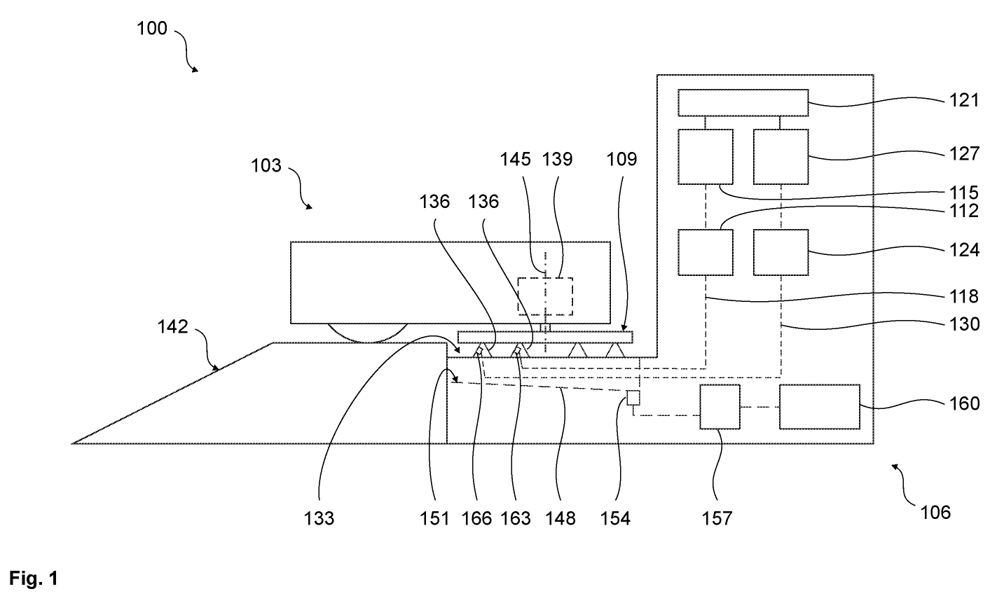

Referring now to the figures of the drawing in detail and first, in particular, to FIG. 1 thereof, there is shown a cleaning and disinfecting system 100 which includes a cleaning appliance 103 and a cleaning and disinfecting station 106 for cleaning or disinfecting, i.e., for hygienizing, a mopping textile 109 of the cleaning appliance 103. The mopping textile 109 in this case is a circular mopping cloth. The cleaning and disinfecting station 106 is accordingly configured as a hygienization station for hygienizing the mopping textile 109, and the cleaning and disinfecting system 100 is a hygienization system. The cleaning appliance 103 is designed here as a cleaning robot, preferably as a mopping robot. The cleaning robot can additionally have a suction function. The cleaning appliance 103 has at least one mopping textile 109, which absorbs dirt during the normal cleaning process of a surface and then docks in the cleaning and/or disinfecting station 106 and, in the present example, is cleaned and disinfected. The cleaning and/or disinfecting station 106 is designed as a base station of the system.

The cleaning and disinfecting station 106 comprises a fluid supply facility 112, or fluid supply 112, for short – configured here as a cleaning fluid supply – which is designed so as to pulse a defined amount of cleaning fluid from a first reservoir 115 and a first supply line 118 onto the mopping textile 109. The cleaning and disinfecting station 106 also comprises a second fluid supply in the form of a disinfecting fluid supply facility 124, or disinfecting fluid supply 124, for short, which is designed so as to pulse a defined amount of disinfecting fluid from a second reservoir 127 and a second supply line 130 onto the mopping textile 109. The (cleaning) fluid supply 112 and the disinfecting fluid supply 124 can each be designed as a pump, for example as a membrane or peristaltic pump, and can be controlled via a control unit 121 in such a manner that the fluid supply takes place at chronologically separate intervals, i.e., in a pulsating manner.

The disinfecting fluid supply 124 here is an in-situ generator for disinfecting fluid. Sodium hypochlorite is particularly suitable as a disinfecting fluid. Further information may be gleaned from our concomitantly filed patent application [Attorney Docket BSH-2025P00153], which is herewith incorporated by reference. By means of the cleaning and disinfecting station 106, the mopping textile 109 can therefore not only be cleaned, but also disinfected.

The cleaning and disinfecting station 106 also comprises a cleaning region 133 for receiving the mopping textile 109, in which mechanical structures 136 are arranged. These mechanical structures 136 are designed so as to mechanically squeeze out the mopping textile 109 by moving the mopping textile 109 over the structures 136. For this purpose, the cleaning appliance 103 has an actuator 139, which is basically configured so as to rotate or oscillate the mopping textile 109 about an axis of rotation 145 in the normal cleaning process, in which a traversed substrate 142 is mopped with the mopping textile 109. This function is used in a cleaning or disinfecting method described herein in order to clean or disinfect the mopping textile 109 by means of the mechanical structures 136 in the cleaning area 133.

The mechanical structures 136 are designed here as projections tapering in the direction of the mopping textile 109 and having a substantially triangular cross-sectional shape, wherein the flanks of these projections guide a fluid that is squeezed out of the mopping textile 109 in a targeted manner into a collecting facility 148 of the cleaning and disinfecting station 106.

The collecting facility 148 is designed as a watertight trough, preferably inclined at the base 151, and has an outlet 154, wherein a discharged fluid that is collected in the collecting facility 148 is guided into the outlet 154 that is arranged at the lowest point of the trough and collected. The dirty fluid is conveyed into a wastewater collecting container 160 via a fluid drain 157, which can be designed, for example, as a pump or vacuum system. The wastewater collecting container 160 is designed as a closed system in order to prevent the escape of fluid vapors. In addition, the wastewater collecting container 160 can be provided with an activated carbon filter or a condensation system in order to bind volatile substances and to reduce odor formation.

According to FIG. 1, two of the mechanical structures 136 each have an opening 163, 166, wherein one of the two openings 163 fluidically connects the cleaning region 133 to the first supply line 118, and wherein the other of the two openings 166 fluidically connects the cleaning region 133 to the second supply line 130. Cleaning fluid is applied to the mopping textile 109 via the first opening 163, wherein disinfecting fluid is applied to the mopping textile 109 via the second opening 166.

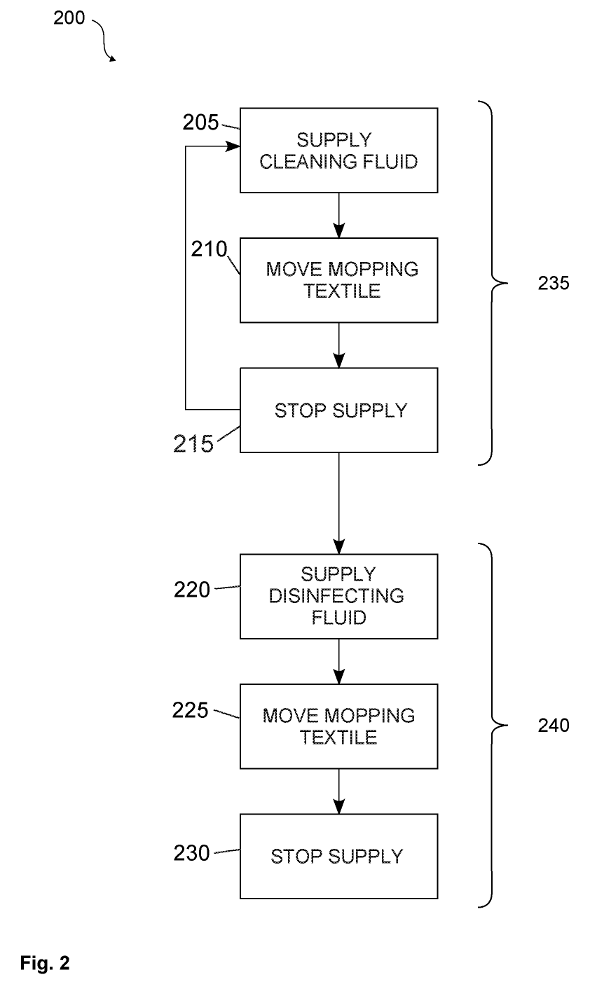

FIG. 2 shows a block diagram of a method 200 for cleaning or disinfecting the mopping textile 109 of the cleaning appliance 103 in the cleaning and disinfecting station 106 according to FIG. 1.

The method 200 is described by way of example on the basis of a cleaning process of the mopping textile 109 with a subsequent disinfecting process. After the cleaning appliance 103, in this case the cleaning robot, has been docked to the cleaning and disinfecting station 106 and the mopping textile 109 is located in the cleaning region 133, in a first step 205, the method 200 begins with a pulsing supply of a defined amount of a cleaning fluid from the first reservoir 115 and via the first supply line 118 to the mopping textile 109 through the fluid supply 112 for a predetermined first period of time. In a second step 210, the mopping textile 109 is then moved over the mechanical structures 136 by rotating the mopping textile 109 by means of the actuator 139, for example, about the axis of rotation 145. Dirt and excess fluid is squeezed out of the mopping textile 109 by the structures 136 and guided into the collecting facility 148, where the fluid is collected.

In a third step 215, the supply of fluid is stopped, while the movement of the mopping textile 109 over the mechanical structures 136 is continued for a predetermined second period of time in order to achieve maximum removal of water from the mopping textile 109. The second period of time is at least five times longer than the first period of time.

During steps 205, 210 and 215, the fluid that is collected in the collecting facility 148 is discharged by means of the fluid draining facility 157 via the outlet 154 into the wastewater collecting container 160. Optionally, the degree of contamination of the discharged fluid can be monitored.

Depending on a degree of contamination of the fluid that is drained or sucked away, steps 205 to 215 can be repeated until the degree of contamination of the fluid falls below a predetermined limit value. A sensor system (not shown here) can be configured so as to detect the degree of contamination of the discharged or extracted fluid. The control unit 121 can control the system 100 based on the collected data.

The combination of pulsing fluid supply, mechanical squeezing and controlled fluid drainage achieves a particularly efficient and fluid-saving disinfection of the mopping textile 109.

After completion of the cleaning process, in a further step 220, a defined amount of disinfecting fluid can be supplied in a pulsing manner from the second reservoir 127 to the mopping textile 109 through the disinfecting fluid supply facility 124 for a predetermined first period of time. In a subsequent step 225, the mopping textile 109 is then moved over the mechanical structures 136, analogously to step 210. Excess disinfecting fluid is squeezed out of the mopping textile 109 by the structures 136 and guided into the collecting facility 148, collected and sucked away into the wastewater collecting container 160 analogously to the above embodiments. In the present invention, "wastewater" is an umbrella term for the excess and often contaminated fluid from the cleaning and disinfecting process, which can be collected in the wastewater collecting container 160, which is designed as a closed tank, and can be emptied at regular intervals, in particular when the container is full.

In a further step 230, the supply of fluid is stopped, while the movement of the mopping textile 109 over the mechanical structures 136 is continued for a predetermined second period of time in order to achieve maximum removal of water from the mopping textile 109. The second period of time is at least five times longer than the first period of time.

The combination of pulsing fluid supply, mechanical squeezing and controlled fluid drainage achieves a particularly efficient and water-saving cleaning of the mopping textile 109.

The method described herein has the advantage that the amount of fluid that is used to clean the mopping textile 109 in the station 106 is reduced. In the same manner, the required amount of disinfecting fluid can be significantly reduced. As a result of the lower input of disinfecting fluid into the mopping textile 109 in terms of volume, the risk of an undesired and possibly harmful gas exceeding the limit value is consequently significantly reduced. In order to further reduce this risk, the fluid draining facility 157 can run on for a certain period of time after the disinfecting process.

After step 230, a conventional cleaning function can be performed with the cleaning appliance 103. When the cleaning function is completed, the method 200 can be implemented again.

It is conceivable for the system 100 to be designed exclusively as a cleaning system for cleaning the mopping textile 109, so that the station 106 is designed as a cleaning station. In this case, only a first part 235 of the method can be performed, which comprises steps 205 to 215.

The system 100 can alternatively be designed only as a disinfecting system for disinfecting the mopping textile 109, so that the station 106 is designed as a disinfecting station. In this case, only a second part 240 of the method 200 is implemented, which comprises steps 220 to 230.

The following is a summary list of reference numerals and the corresponding structure used in the above description of the invention:
100 Cleaning and/or disinfecting system
103 Cleaning appliance
106 Cleaning and/or disinfecting station
109 Mopping textile
112 Fluid supply facility, fluid supply
115 First reservoir
118 First supply line
121 Control unit, controller
124 Disinfecting fluid supply facility, disinfecting fluid supply
127 Second reservoir
130 Second supply line
133 Cleaning region
136 Mechanical structure
139 Actuator
142 Substrate
145 Axis of rotation
148 Collecting facility
151 Base of the collecting facility
154 Outlet
157 Fluid draining facility, fluid drain
160 Wastewater collecting container
163 Opening
166 Opening
200 Method
205 Supplying cleaning fluid
210 Moving mopping textile
215 Stopping supply
220 Supplying disinfecting fluid
225 Moving mopping textile
230 Stopping supply
235 First part
240 Second part

1. A method for cleaning and/or disinfecting a mopping textile of a cleaning appliance in a base station, the method comprising the following steps:
a) supplying, in a pulsating supply of fluid, a defined amount of a cleaning or disinfecting fluid to the mopping textile for a predetermined first period of time;
b) moving the mopping textile over mechanical structures to squeeze dirt and excess fluid out of the mopping textile and discharging the dirt and excess fluid into a collecting facility; and
c) subsequently stopping the supply of fluid while continuing a movement of the mopping textile over the mechanical structures for a predetermined second period of time;
wherein the first period of time is shorter than the second period of time.

2. The method according to claim 1, which comprises, during at least one of step a), step b), or step c), discharging the fluid that is collected by the collecting facility via an outlet into a wastewater collecting container.

3. The method according to claim 1, wherein the second period of time is at least five times longer than the first period of time.

4. The method according to claim 2, which comprises detecting a degree of contamination of the fluid that is discharged into the wastewater collecting container.

5. The method according to claim 4, which comprises repeating steps a) to c) until a detected degree of contamination falls below a limit value.

6. The method according to claim 1, which comprises moving the mopping textile by rotation or oscillation during at least one of step b) or step c).

7. A base station for cleaning and/or disinfecting a mopping textile of a cleaning appliance, the base station comprising:
a fluid supply for feeding, in a pulsating supply, a defined amount of cleaning or disinfecting fluid to the mopping textile;
a collecting facility for collecting fluid; and
a cleaning region having mechanical structures configured to squeeze out dirt and excess fluid by mechanical processing of the mopping textile, and said cleaning region being configured to discharge the dirt and excess fluid into the collecting facility.

8. The base station according to claim 7, wherein said collecting facility includes an outlet for discharging collected fluid into a wastewater collecting container.

9. The base station according to claim 8, which comprises a fluid draining facility configured to discharge fluid that is collected in said collecting facility into said wastewater collecting container via the outlet.

10. The base station according to claim 8, wherein said wastewater collecting container is a closed system preventing fluid vapors from escaping.

11. The base station according to claim 7, wherein said mechanical structures are projections having a substantially triangular cross-sectional shape, and wherein said projections are disposed to come into contact with the mopping textile of the cleaning appliance.

12. The base station according to claim 7, wherein at least one of said mechanical structures is formed with one or more openings for supplying fluid.

13. The base station according to claim 7, wherein a number of said mechanical structures is at least four and is an even number.

14. The base station according to claim 7, wherein said fluid supply is configured for providing and supplying water or cleaning fluid, and further comprising a disinfecting fluid supply configured for providing and supplying disinfecting fluid to the mopping textile.

15. A cleaning and/or disinfecting system, comprising a base station according to claim 7 and a cleaning appliance having at least one movable mopping textile.

16. The cleaning and/or disinfecting system according to claim 15, wherein said mopping textile of said cleaning appliance is configured to rotate or to oscillate.
